(19) United States
(12) Patent Application Publication
(10) Pub. No.: US 2013/0158509 A1
(43) Pub. Date: Jun. 20, 2013

(54) SYSTEM, APPARATUS, AND METHOD FOR DENERVATING AN ARTERY

(71) Applicants: Paul M. Consigny, San Jose, CA (US); Syed Hossainy, Hayward, CA (US)

(72) Inventors: Paul M. Consigny, San Jose, CA (US); Syed Hossainy, Hayward, CA (US)

(21) Appl. No.: 13/329,669

(22) Filed: Dec. 19, 2011

(51) Int. Cl. A61M 37/00; A61B 18/00

(52) U.S. Cl. 604/508; 606/41

(57) ABSTRACT

The present disclosed subject matter is directed to a less invasive surgical system, apparatus and method including a guidable catheter for providing electroporation therapy to a subject suffering from heart or kidney disease. The system includes an electrical generator and an apparatus for denervating an artery. The apparatus includes a catheter having a proximal end, a distal end, a first lumen with a first exit port, a second lumen having a second exit port. The apparatus also includes a first needle including a first electrode. The apparatus also includes a displacement mechanism engaged to the catheter near the proximal end such that the displacement mechanism controls the linear position and angular position of the catheter.

### FIELD OF THE DISCLOSED SUBJECT MATTER

The disclosed subject matter relates to an apparatus, system, and method for denervating an artery. Particularly, the present disclosed subject matter is directed to a less invasive surgical system including a guidable catheter for providing electroporation therapy to a subject suffering from heart or kidney disease.

### DESCRIPTION OF RELATED ART

Elevated nerve signals to and from the kidney are associated with the progression of chronic diseases including heart failure, renal failure and hypertension. In a patient suffering congestive heart failure (“CHF”), the heart progressively fails, and blood flow and pressure will drop in the patient's circulatory system. This results in abnormal activity of the kidney, which itself becomes a principal non-cardiac cause and effect of the disease. During acute heart failure, short-term compensations serve to maintain perfusion to critical organs, notably the brain and the heart that cannot survive prolonged reduction in blood flow. One mechanism by which the body compensates for heart failure is by activating the sympathetic efferent nervous system. This activation results in the local release of norepinephrine and epinephrine that increases cardiac contractility and heart rate, increases venous blood return by increasing peripheral arterial and venous vasoconstriction, and increases blood volume by reducing urine production and by increasing salt retention. However, these same responses that initially aid survival during acute heart failure become deleterious during chronic heart failure. For example, overstimulation of the sympathetic efferents can lead to desensitization. As the heart continues to degrade and blood pressure drops, the kidneys become impaired due to insufficient blood pressure for perfusion. This impairment in renal function ultimately leads to a decrease in urine output. Without sufficient urine output, the body retains fluids, and the resulting fluid overload causes peripheral edema (swelling of the legs), shortness of breath (due to fluid in the lungs), and fluid retention in the abdomen, among other undesirable conditions in the patient. These effects further contribute to a decrease in blood pressure and blood flow, which lead to hypoxia in the body's organs and increases the likelihood of death.

Clinical experience and animal research indicate that an increase in renal sympathetic nerve activity leads to vasoconstriction of blood vessels supplying the kidney, decreased renal blood flow, decreased removal of water and sodium from the body, and increased renin secretion. Accordingly, these conditions may be alleviated by interrupting the nerve signals to and from the kidney to prevent the kidney from contributing to the disease's progression.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods, apparatuses and systems particularly pointed out in the written description and claims hereof, as well as from the appended drawings.

To achieve these and other advantages and in accordance with the purpose of the disclosed subject matter, as embodied and broadly described, the disclosed subject matter includes an apparatus for denervating an artery that includes a catheter having a proximal end, a distal end, a first lumen with a first exit port, a second lumen having a second exit port. The apparatus also includes a first needle including a first electrode. The first needle has a first end and a second end wherein the first end of the first needle is moveable relative to the catheter. The apparatus may also include a second needle including a second electrode, the second needle having a third end and a fourth end wherein the third end of the second needle is moveable relative to the catheter. The apparatus also includes a displacement mechanism engaged to the catheter near the proximal end such that the displacement mechanism controls the linear position of the catheter. The displacement mechanism can alternatively control the angular position of the catheter. In some embodiments, the displacement mechanism can control both the catheter's angular position and linear position. Accordingly, the displacement mechanism enables controlling the linear distance between the first exit port when the displacement mechanism is in a first orientation and the first exit port when the displacement mechanism is in a second orientation. Similarly, the angular distance between the first exit port when the displacement mechanism is in a first orientation and the first exit port when the displacement mechanism is in a second orientation can be controlled. In some embodiments, the linear distance between the first exit port in the first orientation and the first exit port in the second orientation is approximately three millimeters to approximately seven millimeters, and, in some embodiments, the angular distance between the first exit port in the first orientation and the first exit port in the second orientation is approximately twenty degrees to approximately sixty-five degrees. In some embodiments the displacement mechanism is automated. In some embodiments, the linear and angular motion may be effected simultaneously or nonsimultaneously.

The disclosed subject matter also includes a system for denervating an artery. The system includes an electrical generator and an apparatus for denervating an artery. The apparatus includes a catheter having a proximal end, a distal end, a first lumen with a first exit port, a second lumen having a second exit port. The apparatus also includes a first needle including a first electrode. The first needle has a first end and a second end wherein the first end of the first needle is moveable relative to the catheter. The apparatus may also include a second needle including a second electrode, the second needle having a third end and a fourth end wherein the third end of the second needle is moveable relative to the catheter. The apparatus also includes a displacement mechanism engaged to the catheter near the proximal end such that the displacement mechanism controls the linear position of the catheter. The displacement mechanism can alternatively control the angular position of the catheter. In some embodiments, the displacement mechanism can control both the catheter's angular position and linear position. Accordingly, the displacement mechanism enables controlling the linear distance between the first exit port when the displacement mechanism is in a first orientation and the first exit port when the displacement mechanism is in a second orientation. Similarly, the angular distance between the first exit port when the displacement mechanism is in a first orientation and the first exit port when the displacement mechanism is in a second orientation can be controlled. In some embodiments, the linear distance between the first exit port in the first orientation and the first exit port in the second orientation is approximately three millimeters to approximately seven millimeters, and, in some embodiments, the angular distance between the first exit port in the first orientation and the first exit port in the second orientation is approximately twenty degrees to approximately sixty-five degrees. In some embodiments the displacement mechanism is automated. In some embodiments, the linear and angular motion may be effected simultaneously or nonsimultaneously. The generator may generate electromagnetic signals including, but not limited to square waves, sawtooth waves, triangular waves, and sine waves.

To achieve these and other advantages and in accordance with the purpose of the disclosed subject matter, as embodied and broadly described, the disclosed subject matter includes a method for denervating an artery in a subject. One step of the method includes introducing a catheter having an elongate tubular body into an artery. The elongate tubular body has disposed therein a first electrode and a second electrode. Another step of the method includes positioning the catheter proximate to a wall of the artery in a first arterial position, the wall having an adventitia. There is also a delivering step whereby the first electrode and the second electrode are delivered into the adventitia in a first adventitial orientation. There is also an activating step that includes activating a first electroporation cycle. There is also another positioning step whereby the catheter is positioned proximate to the wall in a second arterial position. There is also another delivering step whereby the first electrode and the second electrode are delivered into the adventitia in a second adventitial orientation. In some embodiments, a catheter is introduced wherein the first electrode is disposed on a first needle and the second electrode is disposed on a second needle. In some embodiments, a catheter is introduced wherein the first needle and the second needle is a single needle. In some embodiments, a catheter is introduced wherein the first needle and the second needle are two needles. In some embodiments, the first arterial position and the second arterial position have a linear distance therebetween. In some embodiments the first electrode in the first adventitial orientation and the first electrode in the second adventitial orientation have an angular distance therebetween. In some embodiments, the linear distance is approximately three millimeters to approximately seven millimeters. In some embodiments, the angular distance is approximately twenty degrees to approximately sixty-five degrees. In some embodiments, the steps of positioning the catheter, delivering the electrode or electrodes, and activating the electroporation cycle, are repeated until at least the first electrode traverses at least an angular distance of approximately 360 degrees. In some embodiments, the steps of positioning the catheter, delivering the electrode or electrodes, and activating the electroporation cycle, are repeated until at least the first electrode traverses at least an angular distance of approximately 30 millimeters. In some embodiments, electricity is delivered in the form of approximately 90 electrical pulses at a frequency of approximately four hertz, the pulses having a potential difference of approximately 600 volts and a duration of approximately 100 microseconds. The pulses may have the form of, e.g., square waves, sine waves, sawtooth waves, or triangle waves. In some embodiments, the method further includes a step of delivering a neurolytic agent to the artery. Examples of neurolytic agents include, but are not limited to, phenol alcohol and absolute alcohol. It is envisioned that the disclosed methods may be applied to a subject suffering a disease. Such diseases include but are not limited to heart failure, chronic renal failure, and hypertension. The method may include a single electroporation cycle, two electroporation cycles, or a plurality of electroporation cycles. The method may further include monitoring the blood pressure of the subject.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the apparatus, method and system of the disclosed subject matter. Together with the description, the drawings serve to explain the principles of the disclosed subject matter.

### BRIEF DESCRIPTION OF THE DRAWINGS

FIG. 1 is a schematic representation of a system for denervating an artery in accordance with the disclosed subject matter.

FIG. 2 is a cross section of the distal section of a catheter in accordance with the disclosed subject matter.

FIG. 3 is a cross section of the distal section of a catheter in accordance with the disclosed subject matter.

FIG. 4 is a cross section of the distal section of a catheter in accordance with the disclosed subject matter.

FIG. 5 is a cross section of the distal section of a catheter in accordance with the disclosed subject matter.

FIG. 6A is an illustration of the distal section of a catheter in accordance with the disclosed subject matter.

FIG. 6B is a cross section of a catheter in accordance with the disclosed subject matter.

### DETAILED DESCRIPTION OF THE PREFERRED EMBODIMENT

Reference will now be made in detail to the preferred embodiments of the disclosed subject matter, an example of which is illustrated in the accompanying drawings. The method and corresponding steps of the disclosed subject matter will be described in conjunction with the detailed description of the system.

The methods and systems presented herein may be used for denervating an artery. The disclosed subject matter is particularly suited for denervating the renal artery to alleviate deleterious effects associated with abnormal kidney activity in subjects having, e.g., CHF, renal failure, etc.

Surgical sympathectomy has been used to decrease sympathetic output. Conventional procedures intended to stop the transmission of nerve signals included cutting the sympathetic nerve chain with, e.g., an electrical cautery or scissor, or clipping the sympathetic nerve chain with, e.g., a clip or clamp. An alternative is chemical sympathectomy, a procedure in which a chemical neurolytic agent such as phenol or absolute alcohol is applied to the surgically isolated nerve. Another alternative is electrical sympathectomy, a procedure in which an electric field is applied to the nerve. The present subject matter concerns the local delivery of a chemical neurolytic agent or electricity using a catheter-based procedure. Examples of such approaches include renal denervation, adrenal denervation, and cardiac denervation. Renal denervation is performed by placing a catheter in a renal artery, advancing a needle or needle-based device out of the catheter and through the wall of the renal artery, and then delivering a chemical or electricity into the perivascular space in order to destroy the sympathetic nerves supplying the kidney. This procedure would be performed bilaterally to denervate both kidneys. Adrenal denervation is performed by placing a catheter in the artery or arteries supplying the adrenal gland, puncturing the artery with a needle introduced through a catheter and then use of the needle for the delivery of a chemical or electricity into the perivascular space in order to denervate the adrenal artery. This procedure would be performed bilaterally to denervate both adrenal glands. Cardiac denervation is performed by placing a catheter in the pericardial sac and using a trans-thoracic or trans-atrial approach, and using the catheter to remove the pericardial fluid and replace it with a chemical that will denervate the epicardium, and then using the catheter to remove the chemical and replace the previously collected pericardial fluid. Alternatively, electricity may be delivered into the pericardium to denervate the nerves therein.

It is contemplated that following denervation by above techniques, regrowth of nerves may be encouraged. For example, an implant for regrowing nerves may be added to the denervated area. This would reestablish the denervated nerural system, thereby making the process reversible. For example, a tissue engineered porours PVDF patch, alginate-collagen gel, or gelfoam may be used.

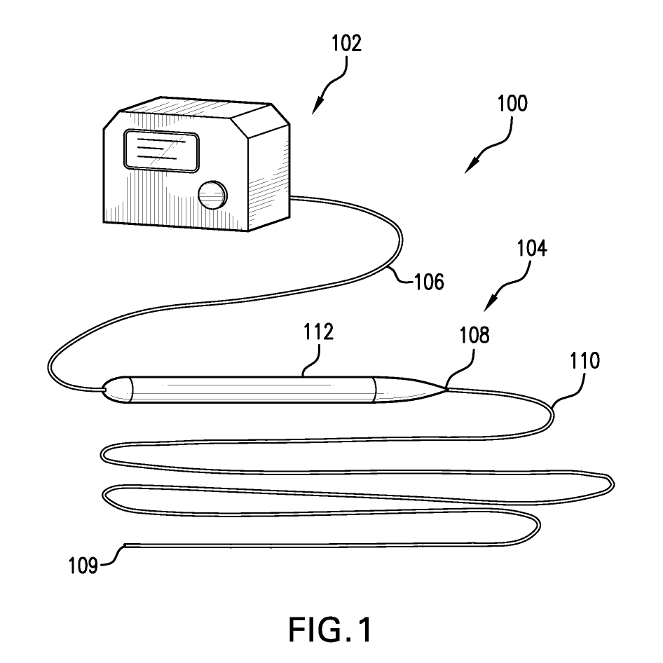

For purposes of explanation and illustration, and not limitation, an exemplary embodiment of the system in accordance with the disclosed subject matter is shown in FIG. 1 and is designated generally by reference character 100, which generally includes an electric signal generator 102 and a catheter assembly 104. Electrical signal generator 102 is preferably of a kind adapted for providing electrical signals suitable for conducting electroporation techniques. In preferred embodiments, generator 102 provides electrical output in the form of sawtooth waves, triangular waves, sine waves, square waves, or the like. An example of a suitable signal generator is an electroporation generator. A particularly suitable generator is the ECM 830 Generator (p/n 45-0052), available from BTX Harvard Apparatus (www.btxonline.com). Catheter assembly 104 includes a catheter 110 having an elongate tubular body, a proximal end 108 and a distal end 109, and a catheter handle having a housing 112. Catheter assembly 104 is connected to generator 102 by wire 106. Catheter assembly 104 is steerable. Techniques for steering catheters are well known in the art. For example, U.S. Pat. No. 7,998,112, which is herein incorporated by reference, discloses mechanisms and techniques for curving the distal end of a catheter to facilitate guiding the catheter through, e.g., a subject's vasculature. As another example, U.S. Pat. No. 7,998,020, which is herein incorporated by reference, discloses a displacement mechanism in the form of an apparatus capable of moving a catheter both linearly along its longitudinal axis and rotationally about its longitudinal axis. In accordance with the present subject matter, catheter assembly 104 includes steering capabilities including, but not limited to, bending, twisting, rotation, advancement, and retraction. The steering mechanism is contained in part within housing 112. Steering controls (not shown) are provided on housing 112, providing a user with the ability to steer catheter 110.

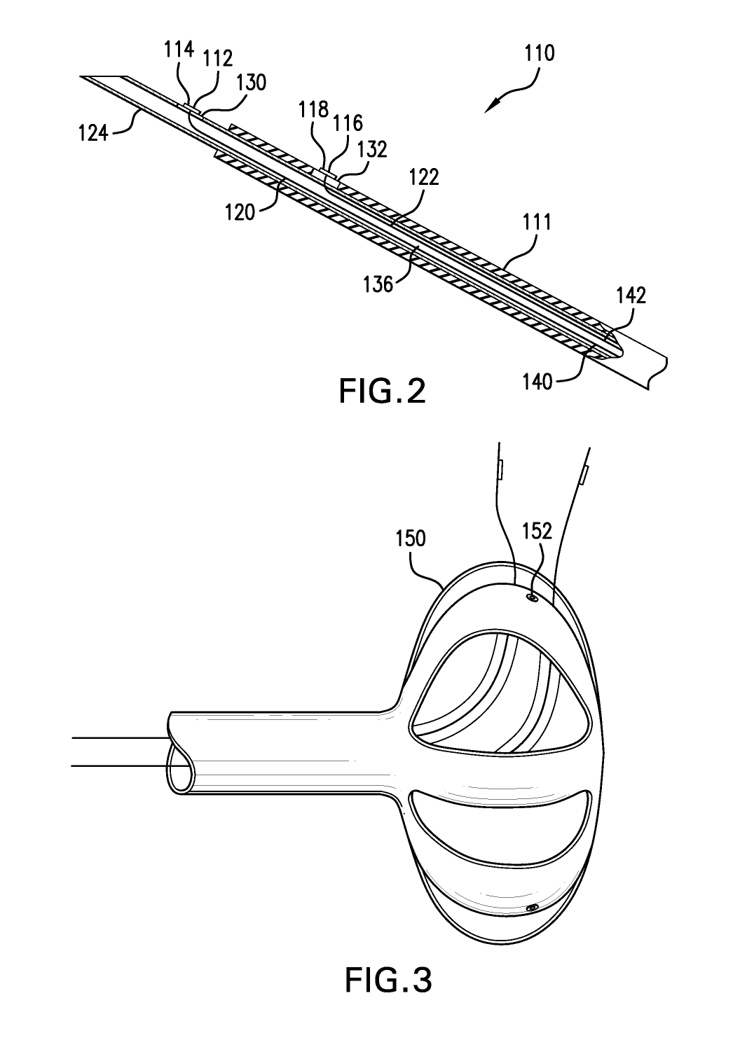

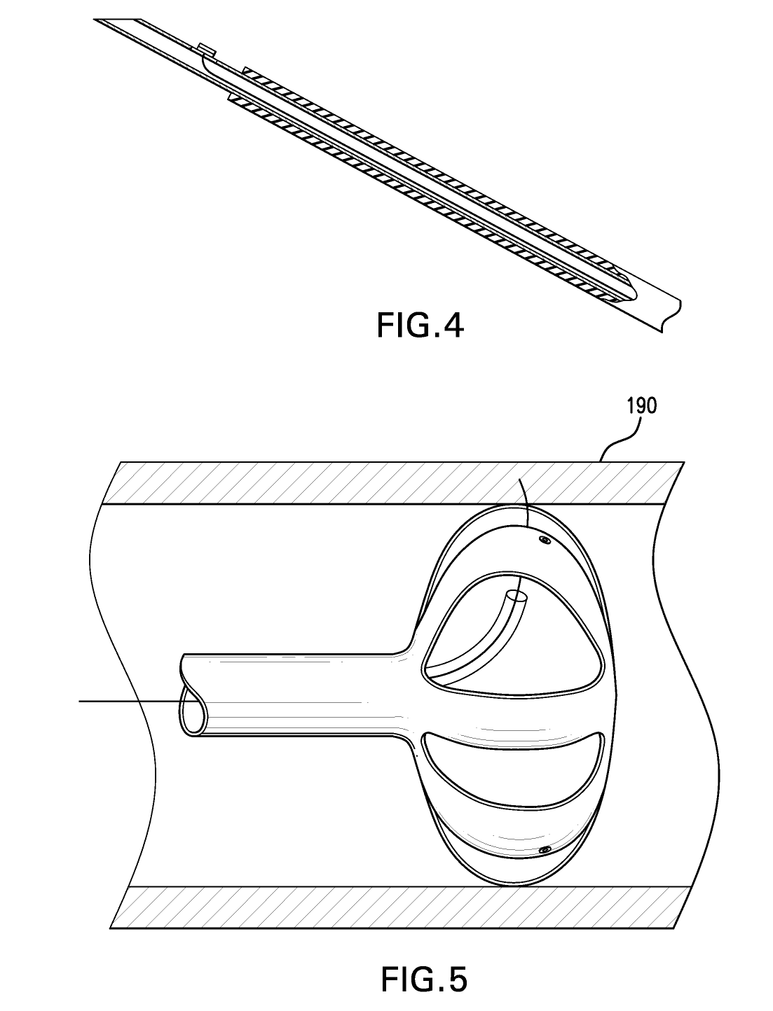

Referring to FIGS. 2 and 3, a distal section of catheter 110 is shown. Catheter 110 includes a catheter body 111, a first lumen 140, a second lumen 142, a first needle 112 having or serving as a first electrode 114 and a second needle 116 having or serving as a second electrode 118. Electrodes 114 and 118 are in electrical communication with insulated wires 120, 122 that are in electrical communication with generator 102. Catheter 110 may additionally include hypotube needle 124. In some embodiments, catheter body 111 is fabricated at least in part from an insulating material. The catheter also includes a first exit port 130 where lumen 140 terminates on the surface of catheter body 111 and a second exit port 132 where lumen 142 terminates on the surface of catheter body 111. Exit ports 130, 132 permit needles 112, 116 and electrodes 114 and 118 to be moved from interior 136 of the catheter to the exterior of the catheter. In some embodiments, the catheter further includes a support system such as cage system 150 to aid in positioning the device by a vessel 190 and advancing the needles. Optionally, a radio opaque marker 152 may be included to aid in device visualization. In some embodiments, two electrodes may be on a single needle, as shown in FIG. 4 and FIG. 5. The needle system may be bipolar insofar as there are two electrodes that may alternatively serve as an anode or cathode.

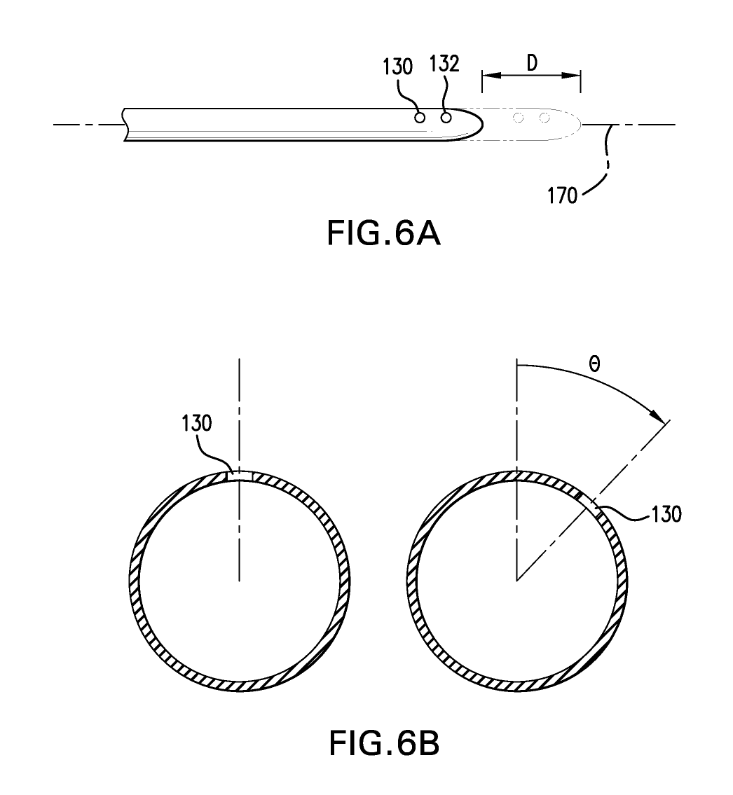

As discussed above, catheter assembly 104 includes a displacement mechanism. The displacement mechanism engages a proximal section 108 of catheter 110. Referring to FIG. 6A, the displacement mechanism enables catheter 110 to be advanced or retracted in a direction parallel to the catheter's longitudinal axis 170. When the displacement mechanism is in a first orientation, catheter 110 is in a first linear position (solid lines), and when the displacement mechanism is in a second orientation, catheter 110 is in a second linear position (dotted lines) that is linearly offset from the first linear position by a linear distance, D. Referring to FIG. 6B, a cross section of catheter 110 taken through exit port 130, the displacement mechanism also enables catheter 110 to be rotated about longitudinal axis 170. When the displacement mechanism is in a third orientation, catheter 110 is in a first angular position, and when the displacement mechanism is in a fourth orientation, catheter 110 is in a second angular position that is angularly offset from the first angular position by an angular distance θ. In some embodiments, the first orientation and the third orientation are the same. In other embodiments, they are different. Similarly, in some embodiments the second and the fourth orientations are the same. In other embodiments they are different. In some embodiments, the displacement mechanism is designed to effect linear and angular motion simultaneously. In other embodiments, the displacement mechanism is designed to effect linear and angular motion nonsimultaneously. In other embodiments, the displacement mechanism includes a user interface that indicates to a user the linear position, linear orientation, angular distance, and/or angular orientation of the catheter. For example, this user interface may comprise, e.g., a moveable knob and detents corresponding to various displacement device orientations. In such an embodiment, the user could move the knob from the first detent to a second detent and understand that he moved the displacement mechanism, e.g., from the first orientation to the second orientation, and that the catheter position changed by D and/or θ. In accordance with the present subject matter, preferred values of D range between approximately three millimeters and approximately seven millimeters, whereas preferred values of θ range between approximately twenty degrees to approximately sixty-five degrees. In some embodiments, the displacement mechanism may be automated so that in response to a user input, the displacement mechanism will automatically move from the first orientation to the second orientation.

Catheter assembly 104 is used with generator 102 to carry out a method of denervating a vessel in a subject's body. Target vessels include any artery or vein within the subject's body where denvervation is desired. It is contemplated, however, that in accordance with the goal of the present subject matter, i.e., alleviating deleterious effects associated with abnormal kidney activity in subjects having, e.g., CHF, the method will be employed in renal arteries, arteries supplying the adrenal gland, and arteries supplying organs that typically receive sympathetic efferents that elicit vasoconstriction. The method includes introducing catheter 104 into a vessel. This introducing step is well known in the art and may include, e.g., a guide catheter. For example, such introduction is often performed in cardiac stent delivery procedures. Catheter 110 is positioned proximate to the wall or adventitia of the vessel in an adventitial orientation. In some procedures where it may be desirable for catheter 110 to press against the vessel wall or adventitia, distal section 109 may be placed into a bent orientation as shown in FIG. 3 and FIG. 5 using one of the steering mechanisms mentioned above. First electrode 114, and in some embodiments, second electrode 118 are delivered into the adventitia by moving needle 120, and in some embodiments, needle 122 through exit port 130, and in some embodiments, exit port 132. In some embodiments, the electrodes are delivered through the wall of the renal artery approximately 0.5 millimeters to 1.0 millimeters into the peradventitial space. Once the electrodes are disposed within the adventitia, generator 102 is activated, thereby subjecting the tissue in the region of the electrodes to an electric field that electroporates the nerve cells therein. In some embodiments, this electroporation cycle is conducted by providing through the electrodes a pulsed electrical signal having a form of, e.g., sawtooth waves, triangular waves, sine waves, square waves. In some embodiments, the pulses have a potential difference of 600 volts. In some embodiments about 90 pulses having a duration of about 100 microseconds duration are delivered at a frequency of about 4 hertz. The electrodes and needles are then retrieved from the artery wall. Finally, the catheter is withdrawn from the vessel.

In many instances, it may be desirable to denervate more, most, or all of the nerves in a vessel, or it may be difficult to sufficiently denervate a vessel from a single application of the electroporation cycle. Accordingly, a plurality of electroporation cycles may be employed. Furthermore, subsequent electroporation cycles may be activated when the needles and electrodes are delivered to a plurality of adventitial positions. For example, when the vessel to be denervated is the renal artery, or the distal main renal artery, or the contralateral renal artery, it will be appreciated that improved denervation will be achieved when catheter 110 is displaced both linearly and angularly to a subsequent adventitial orientation between each electroporation cycle. Specifically, catheter 110 may be displaced three to seven millimeters in a direction along longitudinal axis 170 and twenty to sixty-five degrees about longitudinal axis 170. Once the catheter has been moved to a subsequent adventitial orientation, the needles and electrodes may be reintroduced into the adventitia and generator 102 is reactivated. These steps may be repeated until the vessel is sufficiently denervated. In some embodiments, the catheter may be moved through a plurality of adventitial orientations such that the angular distance traversed is at least 360 degrees and/or the linear distance traversed is at least twenty millimeters. In this manner, the electrodes and needles may be inserted into the vessel wall at points along a path that is, for example, linear, circular, zig-zag, or helical.

In some embodiments, the method includes the additional step of delivering a neurolytic agent to the artery. This step may be performed once, or, in some embodiments, it may be performed before or after each electroporation cycle. Such agents may include, but are not limited to, alcohol, e.g., phenol alcohol, absolute alcohol, or glycerol. Alternatively, butamben, another drug used as a neurolytic, may be used.

The methods described are, in accordance with the present subject matter, particularly suited to be employed on a subject suffering from various diseases, including, but not limited to, heart failure, chronic renal failure, and hypertension. Additionally, it may be desirable to monitor a patient's blood pressure before, during, or after the described procedure because, in accordance with the present subject matter, the apparatus and method are intended to alleviate abnormal kidney function that can lead to decreased blood pressure.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A method for denervating an artery in a subject, comprising:
introducing a catheter having an elongate tubular body into an artery, the elongate tubular body including disposed therein a first electrode and a second electrode;
positioning the catheter proximate to a wall of the artery in a first arterial position, the wall having an adventitia;
delivering the first electrode and the second electrode into the adventitia in a first adventitial orientation;
activating a first electroporation cycle;
positioning the catheter proximate to the wall in a second arterial position; and
delivering the first electrode and the second electrode into the adventitia in a second adventitial orientation.

2. The method of claim 1, further including introducing the catheter wherein the first electrode is disposed on a first needle and the second electrode is disposed on a second needle.

3. The method of claim 2, further including introducing the catheter wherein the first needle and the second needle is a single needle.

4. The method of claim 2, further including introducing the catheter wherein the first needle and the second needle are two needles.

5. The method of claim 1 wherein the first arterial position and the second arterial position have a linear distance therebetween.

6. The method of claim 5 wherein the first electrode in the first adventitial orientation and the first electrode in the second adventitial orientation have an angular distance therebetween.

7. The method of claim 6 wherein the linear distance is approximately three millimeters to approximately seven millimeters.

8. The method of claim 7 wherein the angular distance is approximately twenty degrees to approximately sixty-five degrees.

9. The method of claim 8 further comprising repeating the positioning, delivering and activating steps until the first electrode traverses at least an angular distance of approximately 360 degrees.

10. The method of claim 1 further including the step of delivering a neurolytic agent to the artery.

11. The method of claim 10 wherein the neurolytic agent is phenol alcohol.

12. The method of claim 10 wherein the neurolytic agent is absolute alcohol.

13. The method of claim 1 applied to a subject suffering at least one of heart failure, chronic renal failure, and hypertension.

14. The method of claim 1 further comprising activating a second electroporation cycle.

15. The method of claim 1 further comprising monitoring the blood pressure of the subject.

16. The method of claim 1 further comprising delivering approximately 90 electrical pulses at a frequency of approximately four hertz, the pulses having a potential difference of approximately 600 volts and a duration of approximately 100 microseconds.

17. The method of claim 1 further comprising delivering the pulses as a square wave.

18. The method of claim 1 further comprising delivering the electrodes at points along a helical path.

19. An apparatus for denervating an artery, comprising:
a catheter having a proximal end, a distal end, a first lumen with a first exit port,and a second lumen having a second exit port;
a first needle including a first electrode, the first needle having a first end and a second end wherein the first end of the first needle is moveable relative to the catheter;
a second needle including a second electrode, the second needle having a third end and a fourth end wherein the third end of the second needle is moveable relative to the catheter; and
a displacement mechanism engaged to the catheter near the proximal end such that the displacement mechanism controls the linear position of the catheter.

20. The apparatus of claim 19 wherein the displacement mechanism controls the angular position of the catheter.

21. The apparatus of claim 20 wherein there is a linear distance between the first exit port when the displacement mechanism is in a first orientation and the first exit port when the displacement mechanism is in a second orientation.

22. The apparatus of claim 21 wherein there is an angular distance between the first exit port in the first orientation and the first exit port in the second orientation.

23. The apparatus of claim 22 wherein the angular distance is approximately twenty degrees to approximately sixty-five degrees.

24. The apparatus of claim 23 wherein the linear distance is approximately three millimeters to approximately seven millimeters.

25. The apparatus of claim 19 wherein the displacement mechanism is automated.

26. The apparatus of claim 19 wherein linear motion and angular motion are effected simultaneously.

27. A system for denervating an artery, comprising:
an apparatus, comprising:
a catheter having a proximal end, a distal end, a first lumen with a first exit port, and a second lumen having a second exit port;
a first needle including a first electrode, the first needle having a first end and a second end wherein the first end of the first needle is moveable relative to the catheter;
a second needle including a second electrode, the second needle having a third end and a fourth end wherein the third end of the second needle is moveable relative to the catheter;
a displacement mechanism engaged to the catheter near the proximal end such that the displacement mechanism controls the linear position of the catheter; and
an electrical generator.

28. The system of claim 27 wherein the displacement mechanism controls the angular position of the catheter.

29. The system of claim 28 wherein there is a linear distance between the first exit port when the displacement mechanism is in a first orientation and the first exit port when the displacement mechanism is in a second orientation.

30. The system of claim 29 wherein there is an angular distance between the first exit port in the first orientation and the first exit port in the second orientation.

31. The system of claim 30 wherein the angular distance is approximately twenty degrees to approximately sixty-five degrees.

32. The system of claim 31 wherein the linear distance is approximately three millimeters to approximately seven millimeters.

33. The system of claim 27 wherein the displacement mechanism is automated.

34. The system of claim 27 wherein linear motion and angular motion are effected simultaneously.

35. The system of claim 27 wherein the electrical generator generates square waves.
